(19) United States
(12) Patent Application Publication
(10) Pub. No.: US 2009/0171174 A1
(43) Pub. Date: Jul. 2, 2009

(54) SYSTEM AND METHOD FOR MAINTAINING BATTERY LIFE

(75) Inventor: J. Christopher Kilborn, Winnetka, IL (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/340,973

(22) Filed: Dec. 22, 2008

Related U.S. Application Data
(60) Provisional application No. 61/009,603, filed on Dec. 31, 2007.

(51) Int. Cl. A61B 5/1455; A61B 5/00

(52) U.S. Cl. 600/324; 600/300

(57) ABSTRACT

Embodiments of the present disclosure relate to a patient monitoring system and method. Embodiments may include a patient monitoring device with a battery, one or more charge drawing components, and an operator interface. The operator interface may provide an option to place the patient monitor in a storage mode in which the one or more charge-drawing components draw no or substantially no charge from the battery.

### RELATED APPLICATION

This application claims priority from U.S. Provisional Application No. 61/009,603, filed, Dec. 31, 2007, which is hereby incorporated by reference herein in its entirety.

### BACKGROUND

The present disclosure relates generally to storage of monitoring instruments. In particular, the present disclosure relates to a storage mode for a monitoring instrument that preserves the battery life of the instrument.

This section is intended to introduce the reader to various aspects of art that may be related to various aspects of the disclosed embodiments, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeter. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient.

Pulse oximetry typically utilizes a patient monitoring device that, among other functions, displays information related to patient vital signs and provides an audible and/or visual alarm when changes in the vital signs so warrant. This improves patient care by facilitating continuous supervision of a patient without continuous attendance by a human observer (e.g., a nurse or physician).

Such monitoring devices may typically be powered using AC power, such as from a conventional wall socket, during operation. In addition, such a monitoring device may include one or more batteries to power the device when disconnected from an external power source or in the event of power failure. Typically the batteries are charged during operation of the monitoring device and, therefore, are maintained with sufficient charge for use when needed. However, in the absence of a regular charging current, such as when the monitoring device is transported or stored for an extended period, the batteries may be depleted. In particular, memory components and/or other circuitry in the monitoring device may continue to draw current when the monitoring device is off and/or unplugged from AC power, thereby depleting the charge of the batteries in such storage situations. Battery power may, therefore, be unavailable when it is desired.

### SUMMARY

Certain aspects commensurate in scope with the disclosure are set forth below. It should be understood that these aspects are presented merely to provide the reader with a brief summary of certain embodiments and that these aspects are not intended to limit the scope of the present disclosure. Indeed, various embodiments may encompass a variety of aspects that may not be set forth below.

According to an embodiment, there may be provided a patient monitor that may include a battery, one or more charge-drawing components configured to draw charge from the battery and an operator interface. The operator interface provides an option to place the patient monitor in a storage mode in which the one or more charge-drawing components draw no or substantially no charge from the battery.

According to an embodiment, there may also be provided a method that includes operating a user interface of a patient monitor to place the patient monitor in a storage mode. One or more charge-drawing components of the patient monitor draw no or substantially no charge from a battery of the patient monitor when the patient monitor is in the storage mode.

According to an embodiment, there may also be provided a method that includes providing one or more charge-drawing components in a patient monitor. A battery is provided in the patient monitor. The patient monitor may be placed in a storage mode such that the one or more charge-drawing components draw no or substantially no charge from the battery.

According to an embodiment, there may also be provided a computer-readable medium. The computer-readable medium may include computer-executable instructions for performing actions that include causing one or more charge-drawing components of a patient monitor to draw no or substantially no charge from a battery of the patient monitor.

### BRIEF DESCRIPTION OF THE DRAWINGS

Advantages of this disclosure may become apparent upon reading the following detailed description and upon reference to the drawings in which:

FIG. 1 is a view of a patient monitor in accordance with aspects of an embodiment;

FIG. 2 is a perspective view of a patient monitor in accordance with aspects of an embodiment;

FIG. 3 is a block diagram of exemplary actions associated with storing, charging and using a patient monitor in accordance with aspects of an embodiment; and

FIG. 4 is a view of a multiparameter monitor and exemplary patient monitor in accordance with aspects of an embodiment.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure relates to a patient monitor that may be stored or transported without depleting its battery. In one such embodiment, a patient monitor, such as a pulse oximeter, may be provided that may be configured with a storage or “shelf” mode which reduces or eliminates drain on the battery when the patient monitor is placed in the storage mode.

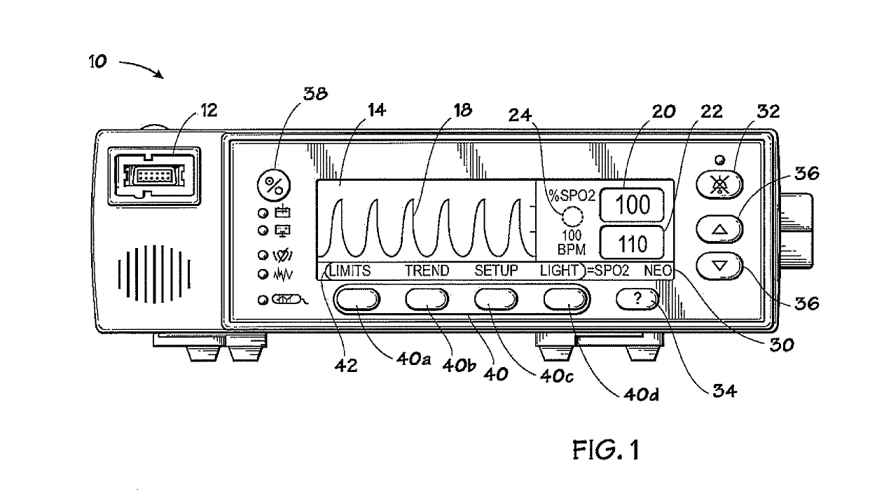

Turning now to FIG. 1, a perspective view of a patient monitor 10 in accordance with one embodiment is provided. For example, the monitor 10 may be a pulse oximeter, such as those available from Nellcor Puritan Bennett LLC, and/or Covidien. The monitor 10 may be processor-based and software-controlled. The software may be stored in memory, such as RAM, ROM, flash, or on an ASIC. Additionally, the monitor 10 may be re-programmable.

In general, the monitor 10 performs functions such as processing physiological data and/or other data received from a patient sensor (discussed below) via a cable connection port 12 that may be configured to communicatively couple with the sensor. The processed data may be displayed in the display window 14, such as a cathode ray tube or liquid crystal display.

For example, the display window 14 may be used to display a plethysmographic (“pleth”) waveform 18, an oxygen saturation 20, and/or a pulse rate 22. The oxygen saturation may be a functional arterial hemoglobin oxygen saturation measurement displayed as units of percentage SpO₂. The pulse rate display 22 may indicate a patient's pulse rate in beats per minute. In one embodiment, the display window 14 may show an initial display immediately after the monitor 10 is turned on that includes the general monitor information, such as the serial number of the instrument, the software version, and so forth. In some embodiments, the display window 14 may also be used to show topic-specific screens, such as a setup and/or configuration screen, a “blip” display that includes pulse amplitude blips, a real-time trend display, and an alarm limit and monitoring mode display.

In addition to displaying physiological information, the monitor 10 may also display information related to alarms and monitor settings. For example, an indicator 24 may be provided to inform an operator that an SpO₂ or pulse reading has been detected which may be above or below an established threshold. Similarly, the display 14 may display an indication of what type of alarm settings, such as adult or neonatal alarm settings, are being employed, such as via indicator 30.

In addition, the monitor 10 may include a number of keys related to the operating functions of the device. The keys may include fixed function keys, such as the alarm silence button 32, help key 34, arrow keys 36 and the power key 38. As will be appreciated by those of ordinary skill in the art, fixed function keys may be configured to control multiple functions or to operate in different manners based upon various factors, such as the duration the key is pressed, the simultaneous activation of other keys, and so forth. For example, an arrow key 36 may be configured to scroll upwards or downwards more rapidly based upon how long the respective key is held down. Similarly, in one embodiment, the help key 34 displays a help screen when held for less than a set time but allows the display contrast to be adjusted via the up and down arrow keys 36 when held for greater than the set time.

The monitor 10 may also include programmable function keys (“soft keys”) 40, and associated soft key icons in the soft key menu 42. Each of the four soft keys 40a, 40b, 40c, and 40d may be pressed to select a corresponding function indicated by the respective soft key icon. For example, the soft key icon menu 42 indicates which software menu items may be selected via the soft keys 40. Pressing a soft key 40 associated with or next to an icon, selects the option.

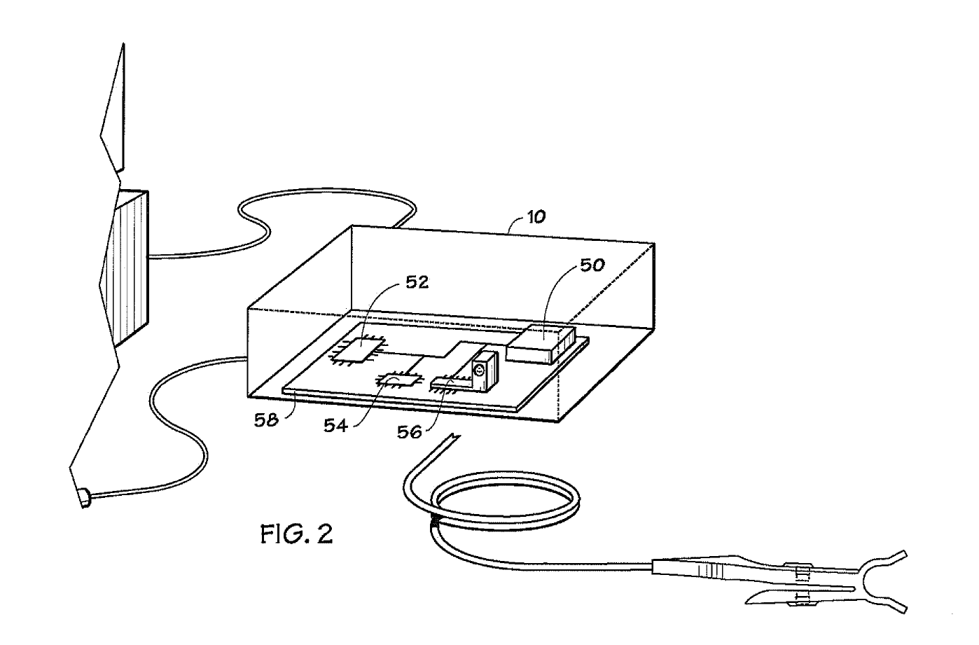

Referring now to FIG. 2, an embodiment of the monitor 10 is depicted which may be run on a battery 50. As depicted, the battery 50 may be internal to the monitor 10 and in such embodiments may or may not be removable. Alternatively, in other embodiments, the battery 50 may be an external battery pack that may be connected to the monitor 10 to power the monitor 10. The battery 50 may consist of one or more than one power cell or charge storing units, which may also be colloquially known as “batteries.” However, as used herein, the term “battery” encompasses one or more than one such charge storing units and generally refers in the aggregate to those power providing units that allow the monitor 10 to operate when not connected to a main AC power supply.

The battery 50 may be connected to components of the monitor 10 that draw power from the battery 50 in at least some circumstances, such as in the absence of an AC current. For example, FIG. 2 depicts charge-drawing components, such as processor 52, memory chip 54, and/or I/O circuitry 56, connected to a system board 58 which may in turn be connected to the battery 50. As one of ordinary skill in the art will appreciate, in the absence of a recharging current, the charge stored by the battery 50 will eventually be depleted by such charge-drawing components over time.

In one embodiment, the depicted monitor 10 may be placed in a shelf or storage mode in which components of the monitor 10 may be prevented from draining the battery 50. In particular, the monitor 10 may be placed in a storage mode for long-term storage (such as storage for greater than three months) or for transport. In one embodiment, the storage mode may be a low or no power mode which places one or more of the charge-drawing circuits, such as the processor 52, memory chips 54, and/or I/O circuitry 56, into a state of no or reduced activity, i.e., a low power or sleep state. In one such embodiment, entry into the storage mode causes one or more routines of power management software running on the monitor 10 to disconnect or substantially disconnect the battery 50 from the system board 58, thereby disconnecting the battery 50 from some or all of the charge drawing components residing on the system board 58.

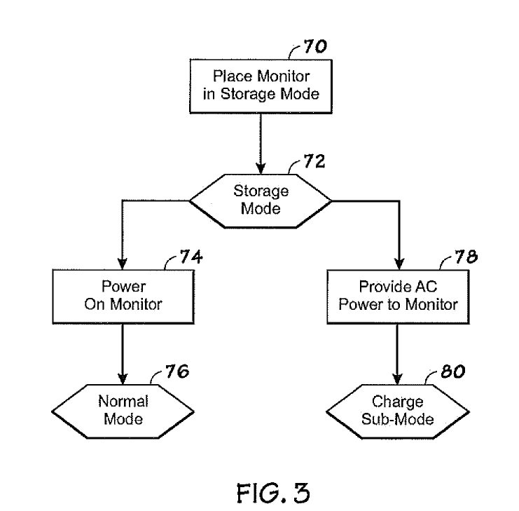

For example, in an embodiment generally described with reference to FIG. 3, an operator places (block 70) the monitor 10 in storage mode (block 72). For example, in one embodiment, the operator selects a storage mode option via a command interface, such as a menu-based user interface of the monitor 10. In such an embodiment, selection of this option causes the monitor 10 to execute those routines associated with the storage mode 72. In another embodiment, the operator may simply press the power on/off button 38 (FIG. 1) to invoke the storage mode 72. In such embodiments, placing the monitor 10 in storage mode 72 causes routines to be executed in the software that controls the power supply or power settings of the monitor. In this example, the executed routines may change the manner and/or frequency in which certain routines, such as I/O routines, operate and/or may otherwise change the power consumption properties of hardware components of the monitor 10, such as to reduce the power consumption of such components.

For example, in one such embodiment, entry into the storage mode 72 places one or more processors 52 of the monitor 10 into a low or no power state, i.e., a sleep mode. Likewise, in some embodiments, watchdog routines (typically routines which reset the operation of the processor 52 in the event of a timer overflow or other indication of a software failure) running on the processor 52 may be turned off or executed at a reduced frequency when in storage mode 72. Further, in one embodiment, a battery 50 may be electrically disconnected from the system board 58 (or otherwise prevented from providing power to the system board 58) by the power management software such that the battery 50 is not drained by charge-drawing components residing on the system board 58. Therefore, in this mode, the processor 52, memory chips 54, I/O circuitry 56, or other power consuming circuits on the system board 58 do not drain the battery 50 or do not drain the battery 50 substantially. In this manner, the battery 50 may be maintained so that the monitor 10 may be operated on battery power even after extended storage.

Referring once again to FIG. 3, various exemplary interactions which may alter or terminate the operation of the storage mode 72 are depicted. For example, in one embodiment the act of powering on the monitor (block 74) causes the monitor 10 to exit storage mode 72 and enter a normal mode 76 of operation in which the software and components of the monitor 10 may not be operating in low or no power states and/or in which the battery 50 may be electrically connected to the main board 58. In one such example, actuating the power button 38 of the monitor 10 causes the monitor 10 to exit storage mode and to begin normal operation suitable for monitoring patient physiological activity.

Likewise, in one embodiment, if the monitor 10 is connected to AC power (block 78) while in storage mode 82, the monitor may enter a battery charge sub-mode 80. In one such embodiment, the processor 52 comes out of the sleep state to charge the battery 50 when the monitor 10 enters such a battery charge sub-mode. In this manner, the battery 50 of the monitor 10 may be charged while in storage without taking the monitor 10 out of storage mode 72. In one embodiment, the I/O circuitry may function normally while the monitor 10 is in a battery charge sub-mode 80. In one embodiment, however, the I/O or other affected circuitry may continue to function in a low or reduced power state until the storage mode 72 is exited, such as by turning the monitor on.

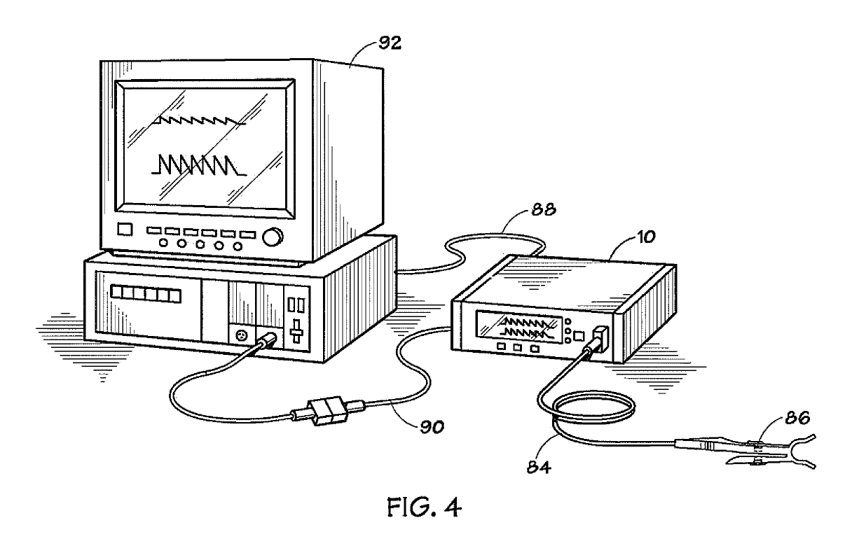

It will be appreciated that a monitor 10 functioning in accordance with the embodiments described herein may be used as part of an aggregate system such as a monitoring system for measuring physiological characteristics of a patient. By way of example, such an exemplary monitor 10 may be used with a sensor 86, as illustrated in FIG. 4, to collect physiological data, such as pulse oximetry data, of a patient, In such an embodiment it should be appreciated that the cable 84 of the sensor 86 may be coupled to the monitor 10 or it may be coupled to a transmission device (not shown) to facilitate wireless transmission between the sensor 86 and the monitor 10. The sensor 86 may be any suitable sensor 86, such as those available from Nellcor Puritan Bennett LLC and/or Covidien. Furthermore, in such an example, to upgrade conventional pulse oximetry provided by the monitor 10 to provide additional functions, the monitor 10 may be coupled to a multi-parameter patient monitor 92 via a cable 90 connected to a sensor input port or via a cable 88 connected to a digital communication port. It should be understood that the functions and functioning of the storage mode described herein may be upgraded through, for example, software upgrades or plug-ins that may enhance or alter the operation of the monitor 10 when in storage mode.

While the disclosed embodiments may be susceptible to various modifications and alternative forms, specific embodiments have been shown by way of example in the drawings and will be described in detail herein. However, it should be understood that the disclosure is not intended to be limited to the particular forms disclosed. For example, other forms of patient monitors, such as other types of spectrophotometric monitors or monitors designed to measure other physiological characteristics, may benefit from the present disclosure. Indeed, the disclosure is to cover all modifications, equivalents and alternatives falling within the spirit and scope of the disclosure as defined by the following appended claims.

What is claimed is:

1. A patient monitor comprising:
a battery;
one or more charge-drawing components capable of drawing charge from the battery; and
an operator interface that provides an option to place the patient monitor in a storage mode in which the one or more charge-drawing components draw no or substantially no charge from the battery.

2. The patient monitor of claim 1, wherein the one or more charge-drawing components generally draw no charge from the battery when the patient monitor is in the storage mode.

3. The patient monitor of claim 1, wherein the one or more charge drawing components comprise a processor, a memory device, and/or an input/output (I/O) interface.

4. The patient monitor of claim 1, wherein the operator interface comprises a display and/or one or more function keys.

5. The patient monitor of claim 1, wherein the storage mode electrically disconnects the battery from the one or more charge drawing components.

6. The patient monitor of claim 1, wherein the storage mode places at least one of the charge-drawing components in a no or relatively low-power state.

7. The patient monitor of claim 1, wherein the patient monitor comprises a pulse oximetry monitor.

8. A method for storing a patient monitor, comprising:
operating a user interface of a patient monitor to place the patient monitor in a storage mode, wherein one or more charge-drawing components of the patient monitor draw no or substantially no charge from a battery of the patient monitor when the patient monitor is in the storage mode.

9. The method of claim 8, wherein operating the user interface comprises selecting a storage mode option via a menu-based interface of the patient monitor.

10. The method of claim 8, wherein the one or more charge-drawing components draw no charge from the battery when the patient monitor is in the storage mode.

11. The method of claim 8, wherein the one or more charge drawing components comprise a processor, a memory device, and/or an input/output (I/O) interface.

12. The method of claim 8, wherein the storage mode places at least one of the charge-drawing components in a no or relatively low-power state.

13. The method of claim 8, wherein the patient monitor comprises a pulse oximetry monitor.

14. The method of claim 8, wherein the storage mode electrically disconnects the battery from the one or more charge-drawing components.

15. One or more tangible media comprising computer-executable instructions for performing actions comprising:
causing one or more charge-drawing components of a patient monitor to draw no or substantially no charge from a battery of the patient monitor.

16. The one or more tangible media of claim 15, wherein the one or more charge drawing components comprise of a processor, a memory device, and/or an input/output (I/O) interface.

17. The one or more tangible media of claim 15, comprising computer-executable instructions for receiving an input from an operator interface that initiates causing the one or more charge-drawing components of the patient monitor to draw no or substantially no charge from the battery of the patient monitor.

18. The one or more tangible media of claim 15, wherein causing the one or more charge-drawing components of the patient monitor to draw no or substantially no charge from the battery of the patient monitor comprises electrically disconnecting the battery from a system board of the patient monitor.

19. The one or more tangible media of claim 15, wherein causing the one or more charge-drawing components of the patient monitor to draw no or substantially no charge from the battery of the patient monitor comprises placing at least one of the charge-drawing components in a no or relatively low-power state.
